(19) United States
(12) Patent Application Publication
(10) Pub. No.: US 2006/0078891 A1
(43) Pub. Date: Apr. 13, 2006

(54) Nucleic acid arrays comprising a set of hybridization parameter determination features and methods for using the same

(76) Inventors: Robert A. Ach, San Francisco, CA (US); Nicholas M. Sampas, San Jose, CA (US); Bo Curry, Redwood City, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/962,286

(22) Filed: Oct. 8, 2004

(51) Int. Cl. C12Q 1/68

(52) U.S. Cl. 435/6

(57) ABSTRACT

Nucleic acid arrays that include a set of hybridization parameter probe features are provided. Also provided are methods of using the subject arrays in hybridization assays. In certain aspects, signals detected from the hybridization parameter probe features may be employed to determine a hybridization parameter of the assay. The subject arrays and methods find use in a variety of different applications. Also provided are computer programming, devices that include the same and kits that find use in practicing the subject methods.

### INTRODUCTION

### Background of the Invention

Array assays between surface bound binding agents or probes and target molecules in solution may be used to detect the presence of particular biopolymeric analytes in the solution. The surface-bound probes may be oligonucleotides, peptides, polypeptides, proteins, antibodies or other molecules capable of binding with target biomolecules in the solution. Such binding interactions are the basis for many of the methods and devices used in a variety of different fields, e.g., genomics (in sequencing by hybridization, SNP detection, differential gene expression analysis, identification of novel genes, gene mapping, finger printing, comparative genomic hybridization, etc.) and proteomics.

One representative array assay method involves biopolymeric probes immobilized in an array on a substrate such as a glass substrate or the like. A solution containing target molecules (“targets”) that bind with the attached probes is placed in contact with the bound probes under conditions sufficient to promote binding of targets in the solution to the complementary probes on the substrate to form a binding complex that is bound to the surface of the substrate. The pattern of binding by target molecules to probe features or spots on the substrate produces a pattern, i.e., a binding complex pattern, on the surface of the substrate which is detected. This detection of binding complexes provides desired information about the target biomolecules in the solution.

The binding complexes may be detected by reading or scanning the array with, for example, optical means, although other methods may also be used, as appropriate for the particular assay. For example, laser light may be used to excite fluorescent labels attached to the targets, generating a signal only in those spots on the array that have a labeled target molecule bound to a probe molecule. This pattern may then be digitally scanned for computer analysis. Such patterns can be used to generate data for biological assays such as the identification of drug targets, single-nucleotide polymorphism mapping, monitoring samples from patients to track their response to treatment, assessing the efficacy of new treatments, etc.

In using nucleic acid arrays, several factors affect hybridization stringency, where representative factors include temperature and salt concentration of the hybridization and wash solutions. Changes in stringency can greatly affect the obtained results from an array experiment. In certain protocols, the stringency of the hybridization and wash steps is determined by noting the temperature of the hybridization oven and the wash solution, and by carefully making the hybridization and wash solutions to particular salt concentrations. However, variations in temperature inside of the hybridization ovens can occur. Furthermore, errors can be produced in the preparation of hybridization and wash solutions. If any of these are present, the actual hybridization stringency of a given array assay may not necessarily be that which is determined based on the above described input parameters.

Accordingly, there is a need for the development of additional methods that can be employed to determine a hybridization parameter, such as temperature or salt condition, of a given hybridization assay, e.g., to provide for a direct measure of the stringency of hybridization of a given assay.

### SUMMARY OF THE INVENTION

Nucleic acid arrays that include a set of hybridization parameter probe features are provided. Also provided are methods of using the subject arrays in hybridization assays. In certain aspects, signals detected from the hybridization parameter probe features may be employed to determine a hybridization parameter of the assay. The subject arrays and methods find use in a variety of different applications. Also provided are computer programming, devices that include the same and kits that find use in practicing the subject methods.

In certain embodiments, methods of determining a hybridization parameter of a nucleic acid array hybridization assay are provided, where the methods include: (a) contacting a nucleic acid array that includes a set of hybridization parameter probe features with a hybridization parameter target sequence; and (b) detecting signals from the set of hybridization parameter probe features to determine the hybridization parameter of said nucleic acid array. In certain embodiments, the set of hybridization parameter probe features includes: (a) a first probe feature that includes a first probe nucleic acid; and (b) a second probe feature that includes a second probe nucleic acid having a sequence that produces a duplex with said hybridization parameter target nucleic acid that is less stable than a duplex formed between the hybridization parameter target nucleic acid and said first probe nucleic acid. The second probe nucleic acid includes at least one nucleotide variant, e.g., in the form of a deletion, insertion or mismatch, compared to the first probe nucleic acid. In certain embodiments, the second probe nucleic acid is shorter than the first probe nucleic acid. In certain embodiments, the set of hybridization parameter probe features further includes a third probe feature comprising a third probe nucleic acid having a sequence that produces a duplex with the hybridization parameter target nucleic acid that is less stable than a duplex formed between the hybridization parameter target nucleic acid and the first probe nucleic acid. In certain embodiments, the determined hybridization parameter is a qualitiative measure, while in other embodiments, the parameter may be a quantitative measure, e.g., temperature, salt concentration, etc. In certain embodiments, the hybridization parameter target nucleic acid is labeled, where the label may be fluorescent. In certain embodiments, the set of hybridization parameter probe features includes a plurality of variant probes, e.g., deletion probes, insertion probse or mismatch probes (or combinations thereof). In certain of these embodiments, the constituent variant probe features of the set may differ from each other in terms of nucleotide variant, e.g., deletion, insertion or mismatch number. In certain embodiments, the plurality of variant probe features includes between about 2 and 10 variant probe features, where in certain embodiments the variant probes have between about 5 and 10 nucleotide variants.

In certain embodiments, a nucleic acid array that includes a set of hybridization parameter probe features, such as those described above, is provided.

Also provided are methods of detecting the presence of a nucleic acid analyte in a sample, where the methods include (a) contacting a nucleic acid array of the invention with a sample suspected of including the analyte under conditions sufficient for binding of that analyte to a nucleic acid probe specific therefore on the array to occur; and (b) detecting the presence of binding complexes on the surface of the array to detect the presence of the analyte in said sample. In certain of these embodiments, the sample also includes a labeled hybridization parameter target nucleic acid. In certain embodiments, the method further includes determining a hybridization parameter of the contacting step using a method of the invention, e.g., as described above. In certain embodiments, the method further includes transmitting a result from a reading of an array from a first location to a second location, where the second location may be a remote location. Also provide are methods that include receiving such a transmitted result.

Also provided are kits for use in a nucleic acid analyte detection assay, where the kits may include an array, as described above, and a hybridization parameter target nucleic acid, as described above.

Also provided are computer-readable mediums having recorded thereon a program that determines a hybridization parameter from signals observed from a set of hybridization parameter probe features of an array.

### BRIEF DESCRIPTION OF THE DRAWINGS

FIGS. 1 and 2 provide a representation of results reported in the Experimental Section below.

### DEFINITIONS

Unless defined otherwise, all technical and scientific terms used herein have the same meaning as commonly understood by one of ordinary skill in the art to which this invention belongs. For the sake of clarity and ease of reference, certain elements are defined below

As used herein, the term “determining” means to identify, i.e., establishing, ascertaining, evaluating or measuring, a value for a particular parameter of interest, e.g., a hybridization parameter. The determination of the value may be qualitative (e.g., presence or absence) or quantitative, where a quantitative determination may be either relative (i.e., a value whose units are relative to a control (i.e., reference value) or absolute (e.g., where a number of actual molecules is determined).

As used herein, the phrase “hybridization parameter” means one of a set of measurable hybridization factors, such as temperature and salt concentration, that define a given hybridization assay and determine its stringency. In certain embodiments, a hybridization parameter of interest is temperature. The term temperature is used in its normal sense to refer to the degree of hotness or coldness of the hybridization assay, and is a measure of the average kinetic energy of the particles in components (and specifically fluid sample) of the hybridization assay, expressed in terms of units or degrees designated on a standard scale, e.g., Celsius. The phrase “salt concentration” refers to the total concentration of salt in a given fluid composition, e.g., expressed in molarity.

In certain embodiments, the term “temperature” is employed to mean “effective temperature”, where it is assumed that the salt concentration is a nominal constant value. In such embodiments, the salt concentration is typically viewed as having a lesser impact than temperature under the ranges of operating conditions of interest. Similarly, in certain embodiments the term “salt concentration” means “effective salt concentration” where it is assumed that the temperature is at a nominal constant value. In such embodiments, the temperature is typically viewed as having a lesser impact than salt concentration under the ranges of operating conditions of interest.

As used herein, the phrase “nucleic acid array hybridization assay” refers to an assay in which a nucleic acid array comprising “probe” sequences is employed. In these assays, a sample of target nucleic acids is first prepared from the initial nucleic acid sample being assayed, where preparation may include labeling of the target nucleic acids with a label, (e.g., such as a member of signal producing system, for example a fluorescent label). Following target nucleic acid sample preparation, the sample is contacted with an array comprising probe features of probe nucleic acid sequences under hybridization conditions, e.g., stringent hybridization conditions, and complexes are formed between target nucleic acids that are sufficiently complementary to probe sequences attached to the array surface. The presence of the resultant complexes is then detected, either qualitatively or quantitatively. Specific hybridization technology which may be practiced to generate the expression profiles employed in the subject methods includes, but is not limited to, the technology described in U.S. Pat. Nos.: 6,656,740; 6,613,893; 6,599,693; 6,589,739; 6,587,579; 6,420,180; 6,387,636; 6,309,875; 6,232,072; 6,221,653; and 6,180,351 and the references cited therein.

The term “nucleic acid” includes DNA, RNA (double-stranded or single stranded), analogs (e.g., PNA or LNA molecules) and derivatives thereof. The terms “ribonucleic acid” and “RNA” as used herein mean a polymer composed of ribonucleotides. The terms “deoxyribonucleic acid” and “DNA” as used herein mean a polymer composed of deoxyribonucleotides. The term “mRNA” means messenger RNA. An “oligonucleotide” generally refers to a nucleotide multimer of about 10 to 100 nucleotides in length, while a “polynucleotide” includes a nucleotide multimer having any number of nucleotides. As such, the term “nucleic acid” includes polymers in which the conventional backbone of a polynucleotide has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides.

The phrase “nucleic acid array” refers to an array of nucleic acid features. An “array,” includes any one-dimensional, two-dimensional or substantially two-dimensional (as well as a three-dimensional) arrangement of addressable regions bearing a particular nucleic acid moiety or moieties (e.g., polynucleotide or oligonucleotide sequences, etc.) associated with that region. The nucleic acids may be covalently attached to the arrays at any point along the nucleic acid chain, but are generally attached at one of their termini (e.g. the 3′ or 5′ terminus).

Any given substrate may carry one, two, four or more or more arrays disposed on a front surface of the substrate. Depending upon the use, any or all of the arrays may be the same or different from one another and each may contain multiple spots (also referred to herein as “features”). A typical array may contain more than ten, more than one hundred, more than one thousand more than ten thousand features, or even more than one hundred thousand features, in an area of less than 20 cm² or even less than 10 cm². For example, features may have widths (that is, diameter, for a round spot) in the range of from about 10 μm to about 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given feature is made up of nucleic acids that hybridize to the same target nucleic acid, such that a given feature corresponds to a particular target nucleic acid. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In certain embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, light directed fabrication methods may be used, as are known in the art. Interfeature areas need not be present particularly when the arrays are made by light directed synthesis protocols.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other). A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

The substrate may be flexible (such as a flexible web). When the substrate is flexible, it may be of various lengths including at least 1 m, at least 2 m, or at least 5 m (or even at least 10 m).

The term “rigid” is used herein to refer to a structure e.g., a bottom surface or a cover that does not readily bend without breakage, i.e., the structure is not flexible.

The phrase “set of hybridization parameter probe features” refers to a collection (i.e., group) of features present on an array surface, where the sequences of the nucleic acid features of the group are chosen such that signals obtained from the features of the set, when used in conjunction with a hybridization parameter target sequence (as defined below), may be employed to determine a hybridization parameter. A given set of hybridization parameter probe features includes a plurality of different features that differ from each other in terms of the sequence of nucleic acids that make up the features. By plurality is meant at least 2, where the number may be 3, 4, 5, 10, 20 or more, and in representative embodiments ranges from 2 to about 25, such as from about 2 to about 20, such as between about 2 and about 10 features. A set of hybridization parameter probe features includes a least a first probe feature and a second probe feature.

The first probe feature includes a sequence that is at least substantially, if not fully complementary over its entire length to a sequence of nucleotides present in a hybridization parameter target sequence. By substantially is meant that, if any mismatches are present between the probe sequence and the target sequence, such do not exceed about 5 nucleotides, such as 3 nucleotides, including 1 nucleotide, where the mismatches, if present, may be present at a terminus of the probe sequence. The term “complementary” is employed to refer to a measure or degree of pairing of complementary nucleotide bases (adenine and thymine, guanine and cytosine) to each other via hydrogen bonds from opposite strands of a double stranded nucleic acid (such as DNA or RNA). As such, complementary sequences are nucleic acid base sequences that can form a double-stranded structure, i.e., duplex, by matching base pairs. For example, the complementary sequence to G-T-A-C is C-A-T-G. Accordingly, two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G, and C of one sequence is then aligned with a T(U), A, C, and G, respectively, of the other sequence. RNA sequences can also include complementary G=U or U=G base pairs. Thus, two sequences need not have perfect homology to be “complementary” under the invention, and in most situations two sequences are sufficiently complementary when at least about 85% (preferably at least about 90%, and most preferably at least about 95%) of the nucleotides share base pair organization over a defined length of the molecule. In those representative embodiments where the nucleic acids of the first probe features are fully complementary to a hybridization parameter target sequence, they are complementary over their entire length to a sequence of nucleotides of the same length in a hybridization parameter target sequence.

In representative embodiments, the second probe feature is made up of nucleic acids of known sequence that are substantially the same as, but not identical to, the sequence of nucleic acids of the first probe feature. As the sequences of the nucleic acids of the second probe feature are not identical to the sequences of the nucleic acids of the first probe feature, there is at least one nucleotide difference between the nucleic acids of the second probe feature as compared to the first probe feature. Nonetheless, the nucleic acids of the second probe feature have a sequence that is substantially the same as the sequence of the nucleic acids of the first probe feature. One sequence is considered to be substantially the same as a second sequence if the sequence similarity between two sequences is at least about 75%, such as at least about 80%, such as at least about 85%, such as at least about 90%, such as at least about 95% or higher. Sequence similarity is calculated based on a reference sequence, which may be a subset of a larger sequence, such as a conserved motif, coding region, flanking region, etc. A reference sequence will usually be at least about 18 nucleotides long, more usually at least about 30 nucleotides long, and may extend to the complete sequence that is being compared. Algorithms for sequence analysis are known in the art, such as BLAST, described in Altschul et al. (1990), J. Mol. Biol. 215:403-10 (using default settings, i.e. parameters w=4 and T=17). In representative embodiments, the BLAST algorithm is employed using default settings to determine sequence similarity.

Because the nucleic acids of the first and second probe features are not identical, there is at least one nucleotide variation between the nucleic acid sequences. The variation may take the form of a deletion or insertion or mismatch, as desired. A second nucleic acid is considered to be a deletion variant of a first nucleic acid if at least one nucleotide residue, wherever positioned in the first nucleic acid, does not appear in the second nucleic acid. A deletion variant may have one or more deletions, e.g., a variant that is missing two nucleotides found in the first sequence is a two deletion variant, etc. A second nucleic acid is considered to be an insertion variant of a first nucleic acid if at least one nucleotide residue, wherever positioned in the second nucleic acid, appears in the second nucleic acid and does not appear in the first. An insertion variant may have one or more insertions, e.g., a variant that has two nucleotides not found in the first sequence is a two-insertion variant, etc. A second nucleic acid is considered to be a mismatch variant of a first nucleic acid if at least one nucleotide residue, wherever positioned in the first nucleic acid, is different in the second nucleic acid. A mismatch variant may have one or more mismatches, e.g., a variant that has two mismatch nts compared to the first sequence is a two-mismatch variant, etc. In certain situations, a given variant may include two or more of deletions, insertions and/or mismatches.

The number of residue variations, e.g., deletions or insertions or mismatches (i.e., combined number of deletions or insertions or mismatches) of a given variant sequence as compared to a first (i.e., reference sequence) may vary and is at least 1 but may be a plurality, i.e., at least 2, such as at least 3, 4, 5 or more, e.g., 10 or more, etc., where the number of variations (where a variation refers to a single nt, and therefore two variant nts is considered a variant sequence with two variations) in certain embodiments does not exceed about 10, e.g., ranges between about 5 and 10 nucleotide variations. The position of the nucleotide variations may be evenly or unevenly spaced along the variant nucleic acid, as desired. A feature of variants of certain embodiments is that they are shorter or longer than the reference nucleic acid. The number of residue mismatches of a given mismatch sequence as compared to a first (i.e., reference sequence) may vary and is at least 1 but may be a plurality, i.e., at least 2, such as at least 3, 4, 5 or more, e.g., 10 or more, etc., where the number of mismatches in certain embodiments does not exceed about 10. The position of the mismatches may be evenly or unevenly spaced along the variant nucleic acid, as desired. A characteristic of mismatch variants of certain embodiments is that they are same length as the reference nucleic acid.

A further aspect of the second, (as well as third, fourth, fifth etc.) probe features of the set of hybridization parameter probe features as compared to the first probe feature is that the nucleic acids of these non-first probe features hybridize to a hybridization parameter target sequence to produce a duplex that is less stable under hybridization conditions, e.g., stringent conditions, than the duplex produced by the nucleic acids of the first probe feature and the hybridization parameter target sequence. As used herein, the term “stable” means resistive to change. As such, a duplex nucleic acid complex is considered stable if the strands of the duplex do not dissociate under stringent hybridization conditions. In representative embodiments, the duplexes of the non-first probe features of the set are less stable than the duplexes of the first probe feature under a given set of conditions by at least about 10-fold, such as by at least about 15-fold, e.g., as determined using the protocol described in Sugimoto, N., Nakano, S-i., Katoh, M., Matsumura, A., Nakamuta, H., Ohmichi, T., Yoneyama, M., Sasaki, M., Biochemistry 34, 11211-11216 (1995); Sugimoto, N., Nakano, M., Nakano, S. Biochemistry 39, 11270-11281 (2000); Freier, S M., Kierzek, R., Jaeger, J A., Sugimoto, N., Caruthers, M H., Neilson, T., Turner, D. H., Proc. Natl. Acad. Sci. USA 83, 9373-9377 (1986).

The phrase “hybridization parameter target sequence” refers to a nucleic acid that is employed in the subject methods as a target nucleic acid that hybridizes to the features of the set of hybridization parameter probe nucleic acids and gives rise to signals from the set of hybridization parameter probe nucleic acids that are employed to determine a hybridization parameter of an assay. The sequence of the hybridization parameter target sequence nucleic acid is chosen so that the target specifically binds to the features of a set of hybridization parameter probe features. In certain aspects, the target is in a sample being assayed, and has a sequence such that it does not detectably bind to other targets in the sample or to probes other than the probes of the corresponding set of hybridization parameter probe features. Hybridization parameter target sequences typically have a sequence that is not present in and will not hybridize to the genome of an organism represented by the corresponding non-hybridization parameter probes on an array. In other words, in most embodiments, if an array contains probes for genes and gene products of a specific species, e.g., humans, the hybridization parameter target sequences in a sample that is intended to be incubated with the array will have a sequence that is not represented in the genome of that species or its products. For example, in embodiments involving samples containing targets from humans, hybridization parameter target sequences may be from yeast, bacteria or any other organism, or may have any other sequence, such that they will not specifically bind to probes for human targets.

A signal refers to any detectable (i.e., identifiable) indicator of the presence or occurrence of an event of interest, e.g., a binding event between two complementary nucleic acids. Signals that are detected in the present invention may vary depending on the signal producing system employed, where the signals may be isotopic, fluorescent, electrical, etc., where in representative embodiments the signals of interest are fluorescent emissions, as is known in the art. The signals observed in the methods of the subject invention are, in certain aspects, generated by a signal producing system. As is known in the art, signal producing systems may vary with respect to the nature of the label system employed therein. Labels of interest include directly detectable and indirectly detectable radioactive or non-radioactive labels such as fluorescent dyes. Directly detectable labels are those labels that provide a directly detectable signal without interaction with one or more additional chemical agents. Examples of directly detectable labels include fluorescent labels. Indirectly detectable labels are those labels which interact with one or more additional members to provide a detectable signal. In this latter embodiment, the label is a member of a signal producing system that includes two or more chemical agents that work together to provide the detectable signal. Examples of indirectly detectable labels include biotin or digoxigenin, which can be detected by a suitable antibody coupled to a fluorochrome or enzyme, such as alkaline phosphatase. In many preferred embodiments, the label is a directly detectable label. Directly detectable labels of particular interest include fluorescent labels. Fluorescent labels that find use in the subject invention include a fluorophore moiety. Specific fluorescent dyes of interest include: xanthene dyes, e.g., fluorescein and rhodamine dyes, such as fluorescein isothiocyanate (FITC), 2-[ethylamino)-3-(ethylimino)-2-7-dimethyl-3H-xanthen-9-yl]benzoic acid ethyl ester monohydrochloride (R6G)(emits a response radiation in the wavelength that ranges from about 500 to 560 nm), 1,1,3,3,3′,3′-Hexamethylindodicarbocyanine iodide (HIDC) (emits a response radiation in the wavelength that ranged from about 600 to 660 nm), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3 (emits a response radiation in the wavelength that ranges from about 540 to 580 nm), Cy5 (emits a response radiation in the wavelength that ranges from about 640 to 680 nm), etc; BODIPY dyes and quinoline dyes. Specific fluorophores of interest include: Pyrene, Coumarin, Diethylaminocoumarin, FAM, Fluorescein Chlorotriazinyl, Fluorescein, R110, Eosin, JOE, R6G, HIDC, Tetramethylrhodamine, TAMRA, Lissamine, ROX, Napthofluorescein, Texas Red, Napthofluorescein, Cy3, and Cy5, and the like.

As used herein, the term “detecting” means to ascertain a signal, either qualitatively or quantitatively.

The term “sample” as used herein refers to a fluid composition, where in certain embodiments the fluid composition is an aqueous composition.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent conditions” refers to conditions under which a probe will hybridize preferentially to its target subsequence, and to a lesser extent to, or not at all to, other sequences. Put another way, the term “stringent hybridization conditions” as used herein refers to conditions that are compatible to produce duplexes on an array surface between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mnM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions that determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). Stringent wash conditions for 60-base oligo probes can include washing in 6×SSC/0.005% Triton X-102 for 10 minutes at 25° C. followed by washing in 0.1×SSC/0.005% Triton X-102 for 5 minutes at 25° C. See Sambrook, Ausubel, or Tijssen (cited below) for detailed descriptions of equivalent hybridization and wash conditions and for reagents and buffers, e.g., SSC buffers and equivalent reagents and conditions.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

As such, the term “hybridization” refers to the formation of a duplex structure by two single stranded nucleic acids due to complementary base pairing. Hybridization can occur between exactly complementary nucleic acid strands or between nucleic acid strands that contain minor regions of mismatch. As used herein, the term “substantially complementary” refers to sequences that are complementary except for minor regions of mismatch, wherein the total number of mismatched nucleotides is no more than about 3 for a sequence about 15 to about 35 nucleotides in length. Conditions under which only exactly complementary nucleic acid strands will hybridize are referred to as “stringent” or “sequence-specific” hybridization conditions. Stable duplexes of substantially complementary nucleic acids can be achieved under less stringent hybridization conditions. Those skilled in the art of nucleic acid technology can determine duplex stability empirically considering a number of variables including, for example, the length and base pair concentration of the oligonucleotides, ionic strength, and incidence of mismatched base pairs. Computer software for calculating duplex stability is commercially available from a variety of vendors.

Stringent, sequence-specific hybridization conditions, under which an oligonucleotide will hybridize only to the exactly complementary target sequence, are well known in the art (see, e.g., Sambrook et al., 2001, Molecular Cloning—A Laboratory Manual, Cold Spring Harbor Laboratory, Cold Spring Harbor, N.Y., incorporated herein by reference). Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the base pairs have dissociated. Relaxing the stringency of the hybridizing conditions allows sequence mismatches to be tolerated; the degree of mismatch tolerated can be controlled by suitable adjustment of the hybridization conditions.

The terms “reference” and “control” are used herein interchangeably to refer to a set of values against which a set of experimentally obtained values may be compared to determine a hybridization pattern of interest. The reference can be in the form of a standardized pattern, e.g., of signals from features obtained under varying values of the hybridization pattern of interest. For example, the reference may be a standardized pattern of signals obtained from a set of hybridization parameter probe features under a series of different experiments in which all but the temperature is held constant, such that one has set of signals in the pattern that are obtained at a plurality of different temperatures. Similarly, the reference may be a standardized pattern of signals obtained from a set of hybridization parameter probe features under a series of different experiments in which all but the salt concentration is held constant, such that one has set of signals in the pattern that are obtained at a plurality of different salt concentrations.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any of the currently available computer-based systems are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### DETAILED DESCRIPTION OF THE INVENTION

Nucleic acid arrays that include a set of hybridization parameter probe features are provided. Also provided are methods of using the subject arrays in hybridization assays. In certain aspects, signals detected from the hybridization parameter probe features may be employed to determine a hybridization parameter of the assay. The subject arrays and methods find use in a variety of different applications. Also provided are computer programming, devices that include the same and kits that find use in practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless th context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. Methods recited herein may be carried out in any order of the recited events which is logically possible, as well as the recited order of events.

All patents and other references cited in this application, are incorporated into this application by reference except insofar as they may conflict with those of the present application (in which case the present application prevails). The citation of any publication is for its disclosure prior to the filing date and should not be construed as an admission that the present invention is not entitled to antedate such publication by virtue of prior invention.

Methods

In one aspect, the subject invention provides methods of determining a hybridization parameter of a nucleic acid array hybridization assay. In these aspects of the invention, a nucleic acid array that includes a set of hybridization parameter probe features is contacted with a fluid sample that includes a hybridization parameter target sequence under hybridization conditions, typically stringent conditions. Following this “hybridization” step, signals from a set of hybridization parameter probe features are then employed to determine a hybridization parameter of the hybridization step, and therefore the hybridization assay.

The nucleic acid arrays employed in embodiments of the invention include at least a set of hybridization parameter probe features. As summarized above, the subject arrays typically include at least two distinct features of nucleic acids made up of nucleic acids that differ by monomeric sequence immobilized on, e.g., covalently or non-covalently attached to, different and known locations on a substrate surface, such as a planar substrate surface. Each feature includes multiple copies of the nucleic acid on the substrate surface, e.g., as a spot on the surface of the substrate. The spots of distinct nucleic acids present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 spots/cm² and usually at least about 100 spots/cm², where the density may be as high as 106 spots/cm²or higher, but will generally not exceed about 105 spots/cm²;

As reviewed above, a feature of the nucleic acid arrays employed in embodiments of the present invention is that they include a set of hybridization parameter probe features. The set includes at least a first probe feature made up of nucleic acid probes that are fully complementary over their entire length to a sequence found in a hybridization parameter target sequence with which the array is designed to be employed. The set also includes at least one additional probe feature which is made up of a nucleic acid probes that have a sequence that is substantially the same as, but not identical to, the sequence of the nucleic acids of the first probe feature of the set. The number of probe features of the set may vary, but is in certain embodiments less than about 20, such as less than about 10 and including from about 2 to about 20, such as from about 2 to about 10. A given set may or may not include multiple identical first probe features, as well as third, fourth, fifth etc., probe features that differ from each other and differ from the first probe feature in a manner analogous to the second probe feature. In certain embodiments, the set of probe features comprises a series of probe features that are different from the first probe feature, where the series of probe features makes up a set of a defined pattern of variations, such as an increasing number of deletions, an increasing number of insertions, an increasing number of mismatches, etc. For example, a set of probe features may include a first probe feature that is fully complementary to the hybridization parameter target sequence and a series of 5 additional probe features each having an increasing number of variant nts, e.g., deletions or insertions or mismatches, e.g., a 5 deletion or insertion probe feature, a 6 deletion or insertion probe feature, a 7 deletion or insertion probe feature, an 8 deletion or insertion probe feature, a 9 deletion or insertion probe feature, and a 10 deletion or insertion probe feature. Each probe feature of the subject arrays is made up of nucleic acid probes, i.e., multiple copies of a given nucleic acid sequence. The total amount of nucleic acid in a given feature may range from about 1×10⁻⁴ pmol to about 0.1 pmol. A length of the nucleic acids making up the probe features may vary, and in certain embodiments ranges from about 5 to about 100 nucleotides, such as from about 10 to about 80 nt, including from about 25 to about 70 nucleotides, e.g., about 50 nt, about 60 nucleotides, etc. As indicated above, the number of variants in the probes of a given set may differ. For example, where the length of the probes is from about 55 to 65 nucleotides, e.g., 60 nucleotides, the number of residue variations, e.g., deletions or insertions or mismatches (i.e., combined number of deletions or insertions or mismatches) of a given variant sequence as compared to a first sequence (i.e., reference sequence) may vary and is at least 1 but may be a plurality, i.e., at least 2, such as at least 3, 4, 5 or more, e.g., 10 or more, etc., where the number of varations (where a variation refers to a single nt, and therefore two variant nts is considered a variant sequence with two variations) in certain embodiments does not exceed about 10, e.g., ranges between about 5 and 10 nucleotide variations. For other probe lengths, the optimal number of variations may differ from the above, where the number is readily determined empirically.

In addition to the set of hybridization parameter probe features, the nucleic acid arrays may include one or more test features that are employed in the detection of nucleic acid analytes in a given assay, as is known in the art.

In aspects of the invention, the nucleic acid array that includes the set of hybridization parameter probe features is contacted under hybridization conditions, e.g., stringent condition, with a sample that includes a hybridization parameter target sequence. In certain embodiments, the hybridization parameter target sequence is labeled. The sample that is contacted with the array may be a test sample in which the hybridization target sequence is provided. Such a test sample may be prepared using any convenient protocol, such as obtaining an initial mRNA sample and adding hybridization parameter target sequence (e.g., in the form of an RNA sequence) thereto (i.e., “spiking in” an RNA template of a hybridization parameter target sequence), followed by labeling the resultant composition using known labeled target generation protocols. Following sample contact with the array, the array is scanned or read to detect the presence, and typically amount (either relative amount or quantitative amount), of duplex nucleic acids in the set of hybridization parameter probe features of the array. The presence (and amount) of duplex nucleic acids in the set of hybridization parameter probe features can be determined using any convenient protocol, e.g., by detecting signals from the set of hybridization parameter probe features of the array, and using the detected signals to determine the presence and/or amount of duplex nucleic acid in the features of the set. (Array hybridization assays, including labeling and detection protocols, are described in greater detail below).

The detected signals (e.g., representing amounts of duplex nucleic acids) are then employed to determine the hybridization parameter of interest. The hybridzation parameter that may be determined may be a qualitative determination or a quantitative determination about the hybridization assay. For example, the determined hybridization parameter may be a simple “yes” or “no” indication that a given hybridization assay has been conducted within one or more predetermined assay parameters, such that the determination provides the operator with with an indication of the overall quality of the assay, or a component thereof, e.g., so that the operator can decide whether to use or discard the assay results (or even use the results knowing that the quality did not meet one or more predetermined criteria). Alternatively, the hybridization parameter can be a quantitative parameter, e.g., the temperature or salt concentration of the sample contacted with the array. The above determinations are made based on the fact that the amount of detected duplex nucleic acids present in a feature of the hybridization parameter probe set is reflective of the hybridization conditions under which hybridization took place. More specifically, the inventors have discovered that amounts of a hybridization parameter target sequence that hybridize to the features of a hybridization parameter probe set, and therefore signals that arise from such features, can be used to determine a hybridization parameter of the conditions under which hybridization took place. Therefore, from the actual detected amount of duplex nucleic acids in the features of the set of hybridization parameter probe features, the hybridization parameter of interest can readily be determined.

Where the methods include detecting signals from labeled target present in the features of the set of hybridization parameter probes, the resultant detected signals may then be employed to determine the hybridization parameter of interest of the assay that has been performed. This determination may be made using any convenient protocol that is capable of using signal data from the set of hybridization parameter probe features of the array, where the signal data may be raw or processed, to determine the hybridization parameter of interest.

The particular protocol employed to determine the hybridization parameter of interest from the input signal data may vary. In certain embodiments, the intensity of the detected signal is employed to make a determination of the relative or absolute amount of labeled target that is bound to the feature. The resultant values for the set of hybridization probe features may then be compared to a reference to determine the hybridization parameter of interest, e.g., the hybridization temperature or salt concentration under the assay conditions.

Programming

Programming for practicing at least certain embodiments of the above-described methods is also provided. For example, algorithms that are capable of determining the hybridization parameter from signal values obtained from a set of hybridization parameter probe features are provided. Programming according to the present invention can be recorded on computer readable media, e.g., any medium that can be read and accessed directly or indirectly by a computer. Such media include, but are not limited to: magnetic tape; optical storage such as CD-ROM and DVD; electrical storage media such as RAM and ROM; and hybrids of these categories such as magnetic/optical storage media. One of skill in the art can readily appreciate how any of the presently known computer readable mediums can be used to create a manufacture that includes a recording of the present programming/algorithms for carrying out the above-described methodology.

Utility

Aspects of the invention find use in a variety of applications, where such applications are generally nucleic acid analyte detection applications (also referred to herein as nucleic acid hybridization assays) in which the presence of a particular nucleic acid analyte in a given sample is detected at least qualitatively, if not quantitatively. Protocols for carrying out such assays are well known to those of skill in the art and need not be described in great detail here. In such methods, a sample of target nucleic acids is first prepared, where preparation may include labeling of the target nucleic acids with a label, e.g., a member of signal producing system. A characteristic of the sample is that it has been modified to include a hybridization parameter target sequence. In certain embodiments, a collection of labeled control targets may be included in the sample, where the collection may be made up of control targets that are all labeled with the same label or two or more sets that are distinguishably labeled with different labels. The sample suspected of comprising the nucleic acid analyte of interest is then contacted with a nucleic acid array that includes a set of hybridization parameter probe features, under conditions sufficient for the analyte to bind to its respective binding pair member that is present on the array. Thus, if the nucleic acid analyte of interest is present in the sample, it binds to the array at the site of its complementary binding member and a complex is formed on the array surface. The presence of this binding complex on the array surface is then detected, e.g. through use of a signal production system, e.g., an isotopic or fluorescent label present on the analyte, etc. The presence of the nucleic acid analyte in the sample is then deduced from the detection of binding complexes on the substrate surface. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER device available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Because a set of hybridization parameter control features and corresponding hybridization parameter target sequence is employed in embodiments of the invention, one can also readily determine a hybridization parameter of the nucleic acid hybridization assay, e.g., by using the methods described above. For example, one can perform a nucleic acid hybridization assay and, from the signals obtained from the set of hybridization parameter probe features, readily determine the temperature of hybridization conditions, assuming other hybridization parameters have been held constant. As such, one can use the methods to provide an independent measure of a hybridization parameter of interest, apart from other measures that may be available. Therefore, the subject methods find use as important quality control (QC) checks in nucleic acid array hybridization assays.

In certain embodiments, the subject methods include a step of transmitting (i.e., communicating) data (i.e., a result) from at least one of the detecting and deriving steps, as described above, to a remote location. The data may be transmitted to the remote location for further evaluation and/or use. Any convenient telecommunications means may be employed for transmitting the data, e.g., facsimile, modem, internet, etc.

Kits

Aspects of the invention include kits that find use in nucleic acid array hybridization assays. The kits may include one or more of the components employed in the methods described above, presented in a kit format. Representative components of interest for kits include, but are not limited to: the nucleic acid arrays, hybridization parameter target sequence, programming for interpreting results, sample preparation reagents, buffers, labels, etc., as described above. The kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay.

Aspects of kit embodiments of the invention may also include instructions for use. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented. In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention.

As with the instructions, the means for obtaining the instructions and/or programming is generally recorded on a suitable recording medium.

The following examples are offered by way of illustration and not by way of limitation.

### EXPERIMENTAL

I. Materials and Methods

### RNA Sample

25 pg of a 472 base polyadenylated “spike-in” RNA transcript was added to 100 ng of human total RNA. The spike-in RNA was designed so that it is not homologous to any sequences in the human genome, and will thus not cross-hybridize to any microarray probes for human RNAs. The mixture of human and spike-in RNAs was amplified and labeled with Cy3 and Cy5 dyes using a T7 RNA polymerase-based method using the Agilent Low RNA Input Fluorescent Linear Amplification Kit (Agilent Technologies, Palo Alto, Calif.) according to the manufacturer's instructions.

### DNA Microarrays

DNA microarrays with 60 base oligonucleotide probes were synthesized by Agilent Technologies, Palo Alto, Calif. The arrays contained 1423 probes for endogenous human genes, as well as a set of 16 probes for the amplified spike-in RNA transcript. Of the probes for the spike-in transcript, four were perfect matches for a 60 base region of the spike-in RNA. Six other spike-in probes contained evenly-spaced deletions of 5, 6, 7, 8, 9, or 10 bases compared to the sequence of the perfect match 60 mer probe. The other six spike-in probes had 5, 6, 7, 8, 9, or 10 base mismatches compared to the perfect match spike-in probes. All probes (perfect match, deletions, and mismatches) were complementary to the same region of the amplified spike-in RNA.

### Hybridization

200 ng of Cy3-labeled sample (the above amplified human+spike-in RNAs) and 200 ng of Cy5-labeled sample were hybridized to fifteen 1900 feature DNA microarrays, using the standard Agilent Technologies hybridization protocol, as described at the document available at the URL made by placing “www.” “chem.agilent.com/scripts/literaturePDF.asp?iWHID=34961”, with the exception that hybridization of five of the arrays were done at 55 degrees Celsius, five arrays were hybridized at 60 degrees Celsius, and five arrays were hybridized at 65 degrees Celsius. After a 16 hour hybridization, arrays were washed in 6×SSC/0.005% Triton X-102 at room temperature for 10 minutes, followed by a wash in 0.1×SSC/0.005% Triton X-102 at room temperature for 5 minutes. Arrays were dried with a nitrogen gun and scanned in the Agilent DNA Microarray Scanner (G2565BA) according to the manufacturer's instructions. Feature analysis was done using the Agilent Feature Extraction Software (G2567AA) (Agilent Technologies, Palo Alto, Calif).

II. Results

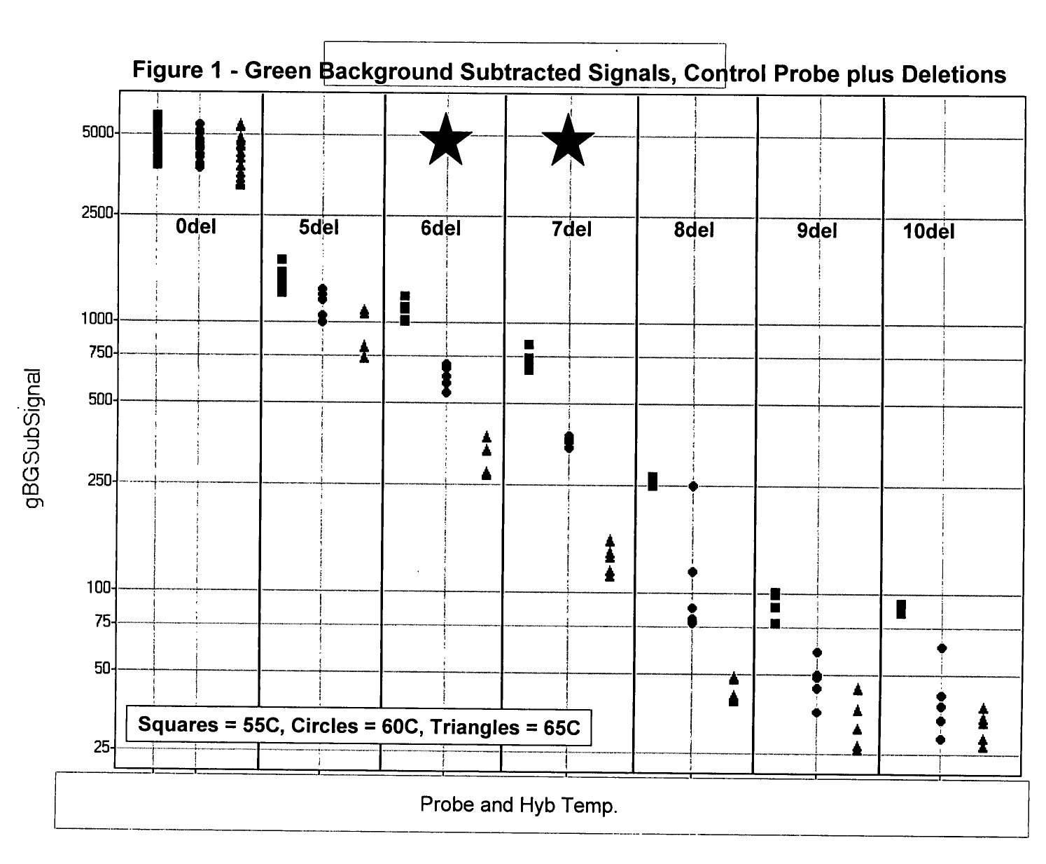

FIG. 1 shows the green (Cy3) background subtracted signals for the spike-in RNA 60 mer probes with perfect matches and 5 to 10 evenly spaced deletions (marked in the figure as 5del, 6del, etc.). The signals are shown for hybridizations done at 55, 60 and 65 degrees Celsius. Each point represents the signal from one of five replicate hybridizations. For the perfect match probes, there are twenty data points at each hybridization temperature, since there were four replicate probes per array. The 55 degree Celsius hyb temperature data points are in blue, the 60 degrees Celsius data points are in green, and the 65 C Celsius data points are in red.

As can be seen, the 6 and 7 base deletion probes give a large signal differential between the different temperatures, such that one could determine the hybridization temperature by examining the strength of the red background subtracted signal of these probes and comparing with the signals from the perfect match probes. Analogous plots and calculations can be done using the red (Cy5) signal as well (data not shown).

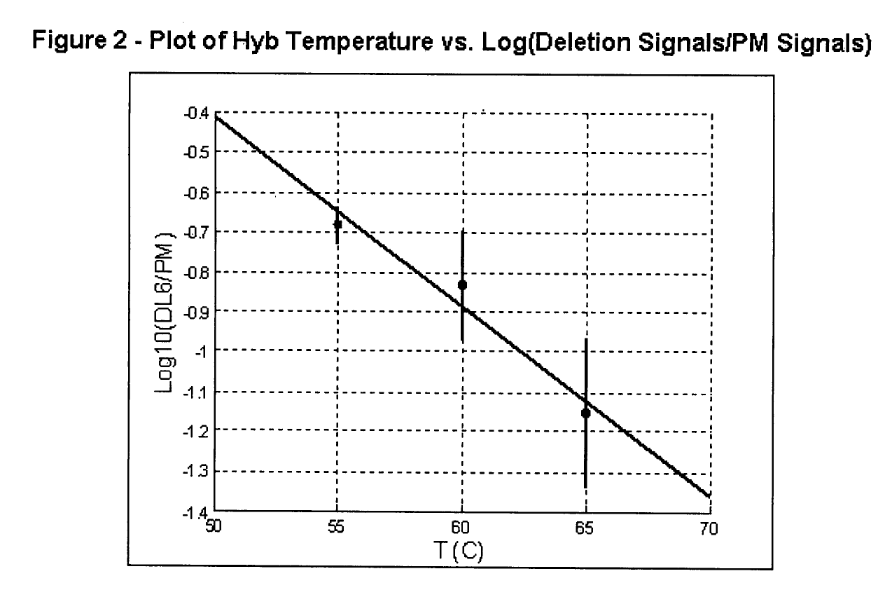

FIG. 2 is a plot of the hybridization temperature versus the log of the ratio of the signal from the 6 base deletions to the signal from the perfect match probes, for the data shown in FIG. 1. The replicates were averaged for this plot. As can be seen the relationship between the hybridization temperature and the log of the signal ratio is linear, allowing calculation of the hybridization temperature if only the signals of the perfect match and deletion probes are known.

Analogous data has been obtained using mismatch probes rather than deletions.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of determining a hybridization parameter of a nucleic acid array hybridization assay, said method comprising:
(a) contacting a nucleic acid array that includes a set of hybridization parameter probe features with a hybridization parameter target sequence; and
(b) detecting signals from said set of hybridization parameter probe features to determine said hybridization parameter of said nucleic acid array.

2. The method according to claim 1, wherein said set of hybridization parameter probe features comprises:
(a) a first probe feature; and
(b) a second probe feature comprising a second probe nucleic acid having a sequence that produces a duplex with said hybridization parameter target nucleic acid that is less stable than a duplex formed between said hybridization parameter target nucleic acid and said first probe nucleic acid.

3. The method according to claim 2, wherein said first probe feature has a higher degree of complementarity to said hybridization parameter target nucleic acid than said second probe feature.

4. The method according to claim 3, wherein said first probe feature has a sequence that is substantially complementary over its entire length to said hybridization parameter target nucleic acid.

5. The method according to claim 4, wherein said first probe feature has a sequence that is completely complementary to said hybridization paramter target nucleic acid over its entire length.

6. The method according to claim 2, wherein said second probe nucleic acid comprises at least one deletion or insertion compared to said first probe nucleic acid.

7. The method according to claim 2, wherein said second probe nucleic acid comprises at least one mismatch compared to said first probe nucleic acid.

8. The method according to claim 2, wherein said second probe nucleic acid is shorter than said first probe nucleic acid.

9. The method according to claim 2, wherein said set of hybridization parameter probe features comprises a third probe feature comprising a third probe nucleic acid that forms a duplex with said hybridization parameter target nucleic acid that is less stable than a duplex formed between said hybridization parameter target nucleic acid and said first probe nucleic acid.

10. The method according to claim 1, wherein said hybridization parameter target is a qualitative measure of said hybridization assay.

11. The method according to claim 1, wherein said hybridization parameter is temperature.

12. The method according to claim 1, wherein said hybridization parameter is salt concentration.

13. The method according to claim 1, wherein said hybridization parameter target nucleic acid is labeled.

14. The method according to claim 13, wherein said label is fluorescent.

15. The method according to claim 1, wherein said set of set of hybridization parameter probe features comprises a plurality of deletion or insertion probe features, wherein constituent deletion or insertion probe features of said set differ from each other in terms of deletion or insertion number.

16. The method according to claim 12, wherein said plurality of deletion or insertion probe features comprises between about 2 and 10 deletion or insertion probe features.

17. A nucleic acid array comprising a set of hybridization parameter probe features.

18. The array according to claim 17, wherein said set of hybridization parameter probe features comprises:
(a) a first probe feature comprising a first probe nucleic acid having a sequence; and
(b) a second probe feature comprising a second probe nucleic acid having a sequence that produces a duplex with said hybridization parameter target nucleic acid that is less stable than a duplex formed between said hybridization parameter target nucleic acid and said first probe nucleic acid.

19. The array according to claim 18, wherein said second probe nucleic acid comprises at least one deletion or insertion compared to said first probe nucleic acid.

20. The array according to claim 18, wherein said second probe nucleic acid comprises at least one mismatch compared to said first probe nucleic acid.

21. The array according to claim 18, wherein said second probe nucleic acid is shorter than said first nucleic acid.

22. The array according to claim 18, wherein said set of hybridization parameter probe features comprises a third probe feature comprising a third probe nucleic acid having a sequence that produces a duplex with said hybridization parameter target nucleic acid that is less stable than a duplex formed between said hybridization parameter target nucleic acid and said first probe nucleic acid.

23. A method of detecting the presence of a nucleic acid analyte in a sample, said method comprising:
(a) contacting a nucleic acid array according to claim 17 having a nucleic acid probe that specifically binds to said nucleic acid analyte with a sample suspected of comprising said analyte; and
(b) detecting the presence of binding complexes on the surface of said array to detect the presence of said analyte in said sample.

24. The method according to claim 23, wherein said sample comprises a labeled hybridization parameter target nucleic acid.

25. The method according to claim 24, wherein said method further comprises determining a hybridization parameter of said contacting step using a method according to claim 1.

26. A method comprising transmitting a result from a reading of an array according to the method of claim 23 from a first location to a second location.

27. The method according to claim 26, wherein said second location is a remote location.

28. A method comprising receiving a transmitted result of a reading of an array obtained according to the method claim 23.

29. A kit for use in a nucleic acid analyte detection assay, said kit comprising:
an array according to claim 17; and
a hybridization parameter target nucleic acid.

30. A computer-readable medium having recorded thereon a program that determines a hybridization parameter from signals observed from a set of hybridization parameter probe features of an array.
